(19) United States
(12) Patent Application Publication
(10) Pub. No.: US 2025/0332141 A1
(43) Pub. Date: Oct. 30, 2025

(54) A PHARMACEUTICAL COMPOSITION COMPRISING AN INHIBITOR OF GLUTAMINE SYNTHETASE ENZYME TO COMBAT ARTEMISININ RESISTANCE IN MALARIA

(71) Applicant: Institute of Life Sciences, Bhubaneswar, Odisha (IN)

(72) Inventors: Viswanathan ARUN NAGARAJ, Bhubaneswar (IN); Govindarajan PADMANABAN, Bangalore (IN); Sourav GHOSH, Bhubaneswar (IN); Rajib KUNDU, Bhubaneswar (IN); Manjunatha CHANDANA, Bhubaneswar (IN); Aditya ANAND, Bhubaneswar (IN)

(73) Assignee: Institute of Life Sciences, Bhubaneswar, Odisha (IN)

(21) Appl. No.: 18/853,042

(22) PCT Filed: Mar. 29, 2023
(86) PCT No.: PCT/IN2023/050301

§ 371 Date: Sep. 30, 2024

(30) Foreign Application Priority Data
Mar. 30, 2022 (IN) 202231018911

(51) Int. Cl. A61K 31/366; A61K 31/198; A61K 31/66; A61K 45/06; A61P 33/06

(52) U.S. Cl. CPC A61K 31/366; A61K 31/198; A61K 31/66; A61K 45/06; A61P 33/06

(57) ABSTRACT

The present disclosure relates to a pharmaceutical composition to combat artemisinin resistance in malaria with the identification of Plasmodium falciparum (Pf) glutamine synthetase (GS) as a drug target. Pf GS is important for maintaining asparagine levels in the parasite and promote protein synthesis. Since ART-resistant parasites undergo fitness loss in amino acid/nutrient-limiting conditions and GS levels are upregulated during ART exposure, targeting Pf GS and asparagine requirement can be harnessed to prevent ART resistance. The present disclosure also provides a method of suppressing the growth of Plasmodium falciparum and a method of treating malaria using GS inhibitors.

### FIELD OF THE INVENTION

The present disclosure relates to a pharmaceutical composition to combat artemisinin resistance in malaria with the identification of Plasmodium falciparum (Pf) glutamine synthetase (GS) as a drug target.

### BACKGROUND OF THE INVENTION

Malaria imposes a serious burden on global health causing enormous morbidity and mortality. As per the World Health Organization (WHO) report, 247 million cases and 619,000 malaria deaths occurred in 2021. Although five Plasmodium species can cause malaria in humans, Plasmodium falciparum (Pf) infections are responsible for more than 90% of the mortality.

Artemisinin (ART) has been used as a frontline antimalarial in combination with other partner drugs for malaria treatment, especially for Plasmodium falciparum infections. It acts mainly on the ring stages by causing rapid clearance of the parasites. However, ART has a short half-life in the circulation and it has to be combined with another partner drug that helps to prevent the emergence of ART-resistance. Recent reports have suggested a delay in the clearance of parasites in patients treated with ART-based combination therapies (ACTs) and emergence of ART resistant strains in WHO South-east Asia and African regions. The mutations that confer resistance to ART have been identified and there are also reports on the emergence of resistance to the partner drugs (1). Therefore, there is an immense need to identify new therapeutic targets and develop pharmaceutical compositions that can help us to combat ART resistance.

Malaria parasites have lost de novo pathways for amino acid biosynthesis and retained only a few transaminases and enzymes functioning at the junction of nitrogen and carbon metabolism (2-5). The asexual stage parasites derive amino acids through hemoglobin (Hb) degradation that occurs in the food vacuole (FV) containing various proteases (6). In vitro cultures of P. falciparum (Pf) can be maintained by providing isoleucine as a sole amino acid in the culture medium, the only amino acid that is absent in human hemoglobin (7). Glutamine is the most abundant amino acid in human blood with plasma concentrations around 0.5 mM (8). The asexual stages and gametocytes can also utilize extracellular glutamine as evident from stable isotope labelling studies (9). In addition, they can acquire glutamine derived from Hb degradation in the FV (7). Glutamine is also abundant in the mosquito hemolymph (10) and it represents 40-60% of the total amino acids in human liver tissue (8). Despite the abundance of glutamine in host milieu and the ability to access Hb-derived and extracellular glutamine, malaria parasite has retained a putative gene for glutamine synthetase (GS) that is conserved across the Plasmodium species infecting humans, primates, rodents and birds (11). It is also known that ART-resistant Pf parasites undergo fitness loss in amino acid/nutrient-limiting conditions and GS levels are upregulated during ART exposure (12,13). In this invention, we provide a new technical advance of targeting GS for inhibiting the growth of Pf. Further, we show that treating ART-resistant PfCam3.IR539T cultures with GS inhibitors could prevent the emergence of viable parasites in ring-stage survival assay (RSA). We claim that targeting GS and asparagine requirement using GS inhibitors can be utilized with the existing ACTs to tackle ART resistance.

### OBJECTS OF THE INVENTION

It is therefore an object of this invention to propose a method of identification of Plasmodium falciparum glutamine synthetase (GS) as a drug target.

Another object of this invention is to demonstrate that GS inhibitors can suppress the growth of Plasmodium falciparum.

Yet another object of this invention is to demonstrate the potential of targeting GS and asparagine requirement to combat falciparum malaria and artemisinin resistance in falciparum malaria.

Another object of this invention is to provide a pharmaceutical composition comprising the combination of L-methionine sulfoximine (MSO)/phosphinothricin (PPT) and artemisinin (ART)/dihydroartemisinin (DHA) can prevent the emergence of ART-resistant parasites and treat ART resistance.

Still another object of the present invention is to provide a method of treating malaria and combating artemisinin resistance, by targeting glutamine synthetase and asparagine requirement using glutamine synthetase inhibitors thereby suppressing the growth of Plasmodium falciparum

These and other objects and advantages of the present invention will be apparent to those skilled in the art after a consideration of the following detailed description.

### SUMMARY OF THE INVENTION

The present invention shows that Plasmodium falciparum (Pf) glutamine synthetase (GS) is enzymatically active and treating in vitro cultures of Pf with inhibitors of GS-methionine sulfoximine (MSO) and phosphinothricin (PPT) can prevent parasite growth. By performing a conditional, mislocalizing, knock sideways for Pf GS, it is shown that GS is required for Pf growth. Targeting PfGS with inhibitors affects the parasite protein synthesis. Glutamine is a major nitrogen resource for the parasite and Pf has several asparagine-rich proteins. The invention shows that the significance of GS arises due to a unique functional requirement of glutamine for the synthesis of asparagine present in these asparagine-rich proteins. Inhibition of parasite protein synthesis at the translation level is involved. By performing ring-stage survival assay (RSA) using tightly synchronized rings of ART-resistant PfCam3.I^{R539T} strain, it is shown that GS can be targeted for ART resistance. In summary, the invention uncovers a new therapeutic target and pharmaceutical composition to combat ART resistance in Pf-the deadliest parasite that is responsible for more than 90% of the malaria mortality.

Accordingly, the present disclosure provides a method of identification of Plasmodium falciparum glutamine synthetase as a drug target.

In another feature of the present disclosure, a method of suppressing the growth of Plasmodium falciparum and a method of treating malaria using GS inhibitors is provided.

In yet another feature of the present disclosure, targeting Plasmodium falciparum glutamine synthetase to inhibit parasite growth is provided.

In still another feature of the present disclosure, a pharmaceutical composition of L-methionine sulfoximine (MSO)/phosphinothricin (PPT) with artemisinin (ART)/dihydroartemisinin (DHA) is shown to tackle ART-resistance.

### BRIEF DESCRIPTION OF THE ACCOMPANYING DRAWINGS

The disclosure may be more fully understood by reading the following detailed description of the embodiment, with reference made to the accompanying drawings as follows:

FIG. 1. illustrates Enzyme activity and inhibition of P. falciparum GS. a) Schematic representation of GS enzymatic reaction. b) Coomassie gel pictures of rPfGS purified using Ni²⁺-NTA resin and their Western blot analysis using anti-his tag antibodies, respectively. Lane M: Protein molecular weight marker (kDa). c) HPLC chromatogram of rPfGS enzyme assays. d) Lineweaver-Burk plots showing Kₘ values of rPfGS for glutamate, ammonia and ATP. rPfGS showed Kₘ values of 0.73±0.21 mM for glutamate, 0.53±0.18 mM for ammonia and 0.75±0.14 mM for ATP, respectively. The Kₘ values (mean±SD) were derived from at least three different protein preparations. e) Effect of MSO and PPT on rPfGS activity. Percentage of activities (mean±SD) with respect to the control (without inhibitor) are shown and the assays were independently carried out with 0.1, 0.5, 1.0 and 2.0 mM concentrations of glutamate. n=3 independent experiments.

FIG. 2. depicts Expression of GS and inhibition of Pf growth by MSO and PPT. a) Western analysis of GS expression in the lysates of Pf rings, trophozoites and schizonts. Equal number of rings (R), trophozoites (T) and schizonts(S) were used from 10 ml of tightly synchronized cultures. b) Immunofluorescence analysis of GS expression in Pf rings (R), trophozoites (T) and schizonts(S). c) Effect of MSO and PPT on in vitro cultures of Pf in RPMI^{-gln} medium. d) Effect of MSO and PPT on in vitro cultures of Pf in RPMI^{Pgln} medium. The data represent mean±SD. For a-d, n=3 independent experiments.

FIG. 3. illustrates Generation of PfGS^{cKS} parasites and performing conditional knock sideways. a) Schematic representation of conditional knock sideways approach to mislocalize PfGS. b) Genomic DNA PCR confirmation for PfGS^{cKS} parasites. Lane 1 and 4:1.76 kb product amplified with GS-specific forward and reverse primers. Lane 2 and 5:2.64 kb product amplified with GS-specific forward and GFP-specific reverse primers to confirm the in-frame fusion. Lane 3 and 6: 1.82 kb product amplified with GS-specific forward and 3′ UTR-specific reverse primer to confirm the integration. Lane M: 1 kb ladder. c) RT-PCR confirmation for PfGS^{cKS} parasites. Lane 1 and 3: 2.51 kb product amplified with GS-specific forward and GFP-specific reverse primers. Lane 2 and 4: 1.63 kb product amplified with GS-specific forward and reverse primers. Lane M: 1 kb ladder. d) Western blot confirmation for GS-FKBP-GFP fusion in PfGS^{cKS} parasites. Upper panel: Confirmation of 120 kDa fusion protein in PfGS^{cKS} parasites with GFP antibody. Middle panel: Confirmation with PfGS antibody. Lower panel: Parasite GAPDH as a loading control. e) Live fluorescence analysis of GS mislocalization in rapamycin-treated PfGS^{cKS+Lyn} asexual stages. Images were captured using 100× objective. Scale bar=5 μM. f) Asexual stage growth analysis of Pf3D7 and PfGS^{cKS} parasites in RPMI^{Ngln}, RPMI^{Pgln} and RPMI^{-gln} medium, and PfGS^{cKS+Lyn} parasites in RPMI^{Pgln} medium. n=at least 3 independent experiments, mean±SD; ***P<0.001, Two-way ANOVA. Rapa-rapamycin.

FIG. 4. depicts Targeting GS in Pf affects asparagine levels and protein synthesis. a) In vitro metabolic labelling of Pf cultures with [³⁵S]-Methionine and-Cysteine. b) SDS-PAGE analysis of protein labelling for Pf parasites. c) In vitro metabolic labelling of Pf cultures for 12 h with [³⁵S]-Methionine and-Cysteine in RPMI^{Pgln} medium. d) SDS-PAGE analysis of protein labelling for Pf proteins. n=3 independent experiments. For a and c, percentage of inhibition (mean±SD) observed for MSO and PPT with respect to solvent control is shown. For b and d, phosphorimager scan was performed after overnight exposure. e) Western analysis of total and phosphorylated eIF2α levels in Pf parasites, respectively. 10 ml of synchronized Pf cultures having rings were treated in vitro with 250 μM MSO for 6 h in RPMI^{-gln} medium. Shorter treatment of Pf rings was preferred since eIF2α phosphorylation occurs at late asexual stages. n=4 independent experiments. f) Quantification of aspartate, glutamate, asparagine and glutamine levels in Pf cultures treated with 50 (n=4) and 250 μM (n=6) MSO. In vitro experiments were carried out in RPMI^{-gln} medium. Relative fold changes of the amino acids with respect to control are plotted after normalizing them with the levels of serine, threonine, histidine, arginine and tyrosine. Box and whisker plots display minimum/maximum points (whiskers), 25^{th}/75^{th} percentile (boxes) and median (centre line).

FIG. 5. illustrates Effect of MSO and PPT on Pf clinical isolates and ART-resistant PfCam3.I^{R539T} strain. a) Effect of MSO and PPT on in vitro growth of Pf (n=7) clinical isolates in RPMI^{-gln}. b) Effect of MSO and PPT on in vitro growth of Pf (n=7) clinical isolates in RPMI^{Pgln}. Growth assessment was carried out based on 3H-hypoxanthine uptake and verified by Giemsa-stained smears. c) Effect of ART/DHA and MSO combination on the growth of ART-resistant PfCam3.I^{R539T} parasites in RSA performed with RPMI^{-gln} medium. (d) Effect of ART/DHA and PPT combination on the growth of ART-resistant PfCam3.I^{R539T} parasites in RSA performed with RPMI^{-gln} medium. e) Effect of ART/DHA and MSO combination on the growth of ART-resistant PfCam3.I^{R539T} parasites in RSA performed with RPMI^{Pgln} medium. (f) Effect of ART/DHA and PPT combination on the growth of ART-resistant PfCam3.I^{R539T} parasites in RSA performed with RPMI^{Pgln} medium. Percentage of growth inhibition of ART/DHA and MSO combination treatment was determined at 96 h with respect to ART/DHA-treated parasites. Percentage of growth inhibition of MSO treatment alone was determined at 96 h with respect to untreated parasites. Similarly, percentage of growth inhibition of ART/DHA and PPT combination treatment was determined at 96 h with respect to ART/DHA-treated parasites. Percentage of growth inhibition of PPT treatment alone was determined at 96 h with respect to untreated parasites. Growth assessment was carried out based on ³H-hypoxanthine uptake and verified by Giemsa-stained smears and flow cytometry. (mean±SD; **P<0.01, ***P<0.001, Two-way ANOVA) n=3 independent experiments.

### DETAILED DESCRIPTION OF THE INVENTION

The detailed description with reference to the accompanying drawings of various exemplary embodiments of the disclosure is described herein. It should be noted that the embodiments are described herein in such details as to communicate the disclosure. However, the amount of details provided herein is not intended to limit the anticipated variations of embodiments; on the contrary, the intention is to cover all modifications, equivalents, and alternatives falling within the spirit and scope of the present disclosure.

It is also to be understood that various substitutions/arrangements/permutations or combinations may be devised that, although not explicitly described or shown herein, embody the principles of the present disclosure. Moreover, all statements herein reciting principles, aspects, and embodiments of the present disclosure, as well as specific examples, intended to encompass equivalents thereof.

The terminology used herein is to describe particular embodiments only and is not intended to be limiting of example embodiments. As used herein, the singular forms “a”, “an” and “the” are intended to include the plural forms as well, unless the context indicates otherwise. It will be further understood that the terms “comprise”, “comprising”, “includes” and/or “including” when used herein, specify the presence of stated features, steps, operations, elements and/or components, but do not preclude the presence or addition of one or more other features, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Glutamine synthetase (GS) is an ancient ubiquitous enzyme that plays a pivotal role in the nitrogen metabolism of prokaryotes and eukaryotes. Glutamine, the product of GS, regulates protein turnover and homeostasis, apoptosis, autophagy, pH homeostatis, cell signalling etc. It is required for nucleotide and hexosamine biosynthesis. Glutamine is also a gluconeogenic and lipogenic precursor, and it serves as an anaplerotic carbon source for TCA cycle controlling cell growth, proliferation and function. A plethora of functions played by glutamine renders it as a versatile and the most abundant amino acid, not only in humans but also in mosquitoes. While the coevolution of malaria parasite with human and mosquito hosts, and the parasitic niche that is conducive for acquiring amino acids from the host, have resulted in the loss of de novo pathways for amino acid biosynthesis, the parasite has retained a gene for GS. This disclosure shows that Pf GS is enzymatically active and it is inhibited by the GS inhibitors-MSO and PPT. MSO and PPT can inhibit the parasite growth in in vitro Pf cultures indicating that PfGS can serve as a drug target. A conditional, mislocalizing, knock sideways performed for Pf GS show that GS is required for Pf growth.

Glutamine serves as an amide donor for asparagine synthesis and Pf proteins are asparagine-rich. Pf parasites treated with GS inhibitor display a prominent decrease in the asparagine levels and undergo an arrest in protein synthesis. Although the results show that PfGS supplies glutamine as an amide donor for asparagine synthesis, they do not exclude the requirement of glutamine for other metabolic pathways. Inhibition of GS in the RSA performed for ART-resistant PfCam3. I^{R539T} strain exposed to ART/DHA prevents the emergence of viable parasites. The exposure of ART-resistant PfCam3.I^{R539T} strain to the combination of ART/DHA with MSO/PPT for a short duration of 6 h leads to decrease in the parasite survival. Hence, the asparagine-rich nature of Pf proteins and the requirement of glutamine as a nitrogen source for asparagine and protein synthesis can be harnessed to target ART-resistance. Inhibition of PfGS by MSO and PPT can offer a platform to develop new inhibitors that are highly specific for PfGS providing better efficacy and favourable therapeutic index for clinical use. The disclosure therefore shows that PfGS can serve as a drug target for falciparum malaria and GS inhibitors can be used for a new pharmaceutical composition with the existing artemisinin-based combination therapies (ACTs) to combat ART resistance.

Accordingly, the present disclosure provides provides a pharmaceutical composition for combating drug resistance in Plasmodium falciparum, comprising inhibitor of glutamine synthetase enzyme and artemisinin or dihydroartemisinin.

In an embodiment of the present disclosure, the inhibitor of glutamine synthetase enzyme is selected from L-Methionine sulfoximine or Phosphinothricin and the inhibitor is in the range of 50 μM to 2 mM in in vitro Plasmodium falciparum cultures. The inhibitor shall be present in the range of 0.1-100 mg/kg of the total body weight in the pharmaceutical composition. The dose of artemisinin, dihydroartemisinin are as per WHO-recommendation. The dose of partner drugs is also as per the WHO recommendation. The inhibitor of glutamine synthetase irreversibly inhibits glutamine synthetase activity leading to a decrease in the production of asparagine and protein synthesis.

In another embodiment of the present disclosure, a method of suppressing the growth of Plasmodium falciparum, by targeting glutamine synthetase and asparagine requirement by providing the pharmaceutical composition comprising an inhibitor of glutamine synthetase enzyme and artemisinin or dihydroartemisinin, is provided.

The present disclosure also provides a method of treating malaria and combating artemisinin resistance, by targeting glutamine synthetase and asparagine requirement using glutamine synthetase inhibitors along with the existing artemisinin-based combination therapies selected from artesunate and amodiaquine; artesunate and mefloquine; artesunate and sulfadoxine-pyrimethamine; artemether and lumefantrine; dihydroartemisinin and piperaquine; artesunate and pyronaridine and/or other antimalarials.

### EXAMPLES

Unless defined otherwise, all technical and scientific terms used herein have the same meaning as commonly understood to one of ordinary skill in the art to which this disclosure belongs.

Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Example 1

### Cloning, Over-Expression and Purification of Recombinant Pf GS

The Plasmodium falciparum used in the present disclosure is obtained from: Pf3D7 cultures-Source: Malaria Research and Reference Reagent Resource Center (MR4), ATCC Manassas Virginia; Geographical origin: Amsterdam, Netherlands. ART-resistant PfCam3.I^{R539T} strain-Source: Malaria Research and Reference Reagent Resource Center (MR4), ATCC Manassas Virginia; Geographical origin: Battambang province, western Cambodia.

cDNA sequence of PfGS (PF3D7_0922600) was retrieved from PlasmoDB (https://plasmodb.org/plasmo/app). Total RNA from Pf was isolated using RNeasy Mini Kit (Qiagen, 74104) according to the manufacturer's protocol. cDNA synthesis was carried out with 1 μg of total RNA using RevertAid Reverse Transcriptase (Thermo Fisher Scientific, EP0442), followed by PCR with Phusion High-Fidelity DNA Polymerase (New England Biolabs, M0530). The following were the forward (F) and reverse (R) primers used: PfGS (F) of Sequence ID No.1: 5′-GCCAGGATCCATGAAGTCCGTGAGTTTTTCAAATAATGC-3′; PfGS (R) of Sequence ID No. 2: 5′-GCCCAGATCTCTAACATTCATAATATAAGTGATAATCATAAGCG-3′. The restriction sites used for cloning are underlined. cDNA product was digested with the respective restriction enzymes and cloned into pRSETA plasmid (Thermo Fisher Scientific). Recombinant protein expression was carried out in E. coli Rosetta2DE3pLysS strain (Novagen). In brief, E. coli Rosetta2DE3pLysS cells transformed with recombinant plasmid was grown to an A₆₀₀ of 1.0 at 30° C. and the protein induction was carried out at 18° C. for 12 h using 1 mM isopropyl-β-D-thiogalactoside (IPTG) (MP Biomedicals, 11IPTG0001). The recombinant protein fused with 6xHis tag was purified using Ni²⁺-NTA Agarose resin (Qiagen, 30210). In brief, the bacterial cell pellets expressing the recombinant protein was resuspended in lysis buffer containing 50 mM Tris pH 8.0, 500 mM NaCl, 20% glycerol, 0.01% Triton X-100, 1 mM dithiothreitol and protease inhibitors, sonicated and centrifuged at 43,000 g for 1 h. The supernatant was separated and loaded onto a column packed with Ni²⁺-NTA resin and washed sequentially with lysis buffer containing 1, 10 and 50 mM imidazole. The recombinant protein was then eluted with lysis buffer containing 150 mM imidazole. The purified protein was then dialyzed against 50 mM Tris pH 8.0, 50 mM NaCl and 20% glycerol. Protein estimation was carried out using Pierce BCA Protein Assay Kit (Thermo Fisher Scientific, 23225). The total yield of recombinant GS was around 0.25-0.50 mg per litre of bacterial culture.

### Example 2

### Enzyme Assays

Enzyme assays were carried out by performing HPLC analysis for glutamine formation as well as by quantifying the release of inorganic phosphate (Pᵢ). The enzyme assays were carried out at 37° C. for 1 h in a total volume of 25 82 l and the assay mixtures contained 50 mM Tris buffer pH 8.0 with 4 mM glutamate, 50 mM NaCl, 50 mM NH₄Cl, 10 mM ATP and 50 mM MgCl₂ and 0.5-1.0 μg of recombinant protein. The amino acids were extracted by vortexing the assay mixtures with 50 μl of water and 375 μl of acetonitrile, followed by incubation in ice for 30 min and centrifugation at 20,000 g for 20 min at 4° C. The supernatant was collected, lyophilized and dissolved in 50 μl of water. 2.5 μl of the sample was subjected to pre-column derivatization with OPA reagent (Agilent, 5061-3335) by programming the auto-sampler and the separation of amino acids was carried out in Agilent 1260 Infinity HPLC System (Agilent Technologies) using a Poroshell 120 HPH-C18 column (4.6 mm×100 mm×2.7 μm) as per the manufacturer's protocol. Mobile phase consisted of Solvent A: 10 mM Na₂HPO₄, and 10 mM Na₂B₄O₇, pH 8.2 and solvent B: methanol: acetonitrile: water (45:45:10 v/v). OPA-derivatized amino acids were detected using 1260 Infinity II Fluorescence Detector with excitation of 340 nm and emission of 450 nm. Amino acid standards (Agilent, 5061-3330) were used to determine the retention time. The standard curves were generated for glutamine, and the assay products were also confirmed by spiking with glutamine. For every molecule of glutamine formed, one molecule of ATP is hydrolysed to ADP and Pᵢ. Therefore, the release of Pᵢ was quantified as an indirect way of measuring glutamine synthesis and the activities were found to be comparable with HPLC assays. For Pᵢ assays, the assay mixtures incubated at 37° C. for 1 h were diluted with water to 200 μl, followed by the addition of 30 μl of phosphate reagent of Phosphate Assay Kit (Abcam, ab65622). After vortexing, the absorbance was measured at 650 nm as per the manufacturer's protocol. Individual glutamate-and protein-omitted reactions were used as controls for all the assays.

### Example 3

In Vitro Maintenance of P. falciparum Cultures

Routine maintenance of Pf3D7 cultures were carried out at 37° C. in RPMI-1640 medium (Gibco, 23400-013) containing 0.5% AlbuMAX-II (Thermo Fisher Scientific, 11021037) using O^{+ve} RBCs of 5% hematocrit under 90% N₂, 5% O₂ and 5% CO₂. The parasites were subcultured with fresh RBCs when the culture parasitemia reached around 2-5%. Synchronization of Pf cultures was carried out with 5% D-sorbitol (w/v) (Sigma-Aldrich, 240850). Parasites were isolated by treating the infected RBC pellet with equal volume of 0.15% saponin in PBS (w/v) (Sigma-Aldrich, S4521) followed by centrifugation at 10,000 g for 10 min at 4° C. The isolated parasites were washed thoroughly with ice cold PBS for four times to remove the carry-over of proteins from RBC lysates. ART-resistant PfCam3.I^{R539T} strain was also maintained in a similar fashion. Giemsa-stained smears were routinely prepared to examine the parasite growth and parasitemia. Parasite growth assessment for experiments was carried out by Giemsa-stained smears, flow cytometry and ³H-hypoxanthine uptake. Flow cytometry was performed by staining the infected RBCs with 0.5× SYBR Green I (Thermo Fischer Scientific, S7563) in HBSS buffer pH 7.4 containing 2% FBS (Gibco, 10270106) for 30 min at 37° C. The data was acquired using BD LSRFortessa (BD). ³H-hypoxanthine uptake assays were performed by adding ³H-hypoxanthine (American Radiolabeled Chemicals, Inc., ART 0266) (5 μCi per ml culture volume) to the cultures. The labelled cultures were then washed with RPMI-1640 medium, lysed in water and harvested on glass fibre filters using FilterMate cell Harvester (PerkinElmer). After washing with water, the glass fibre filters were dried and placed in Ultima Gold XR scintillation cocktail (PerkinElmer, 6013111). The radioactive counts were measured using MicroBeta² Microplate Counters (PerkinElmer). Glutamine-free RPMI was procured from Thermo Fisher Scientific (Gibco, 42401-018) and for experiments carried out at physiological concentrations of glutamine, glutamine-free RPMI-1640 was reconstituted with 0.5 mM glutamine (Sigma-Aldrich, G8450).

### Example 4

### Immunofluorescence Analysis

Immunofluorescence analysis was carried out by fixing the cells with 4% paraformaldehyde and 0.0075% glutaraldehyde, followed by permeabilization with 0.1% Triton X-100 and subsequent treatment with 0.1 M glycine. Blocking was performed with PBS containing 2% BSA for 3 h. The incubation for primary antibodies was carried out in the same blocking buffer for 6 h, followed by the addition of secondary antibody for 3 h. Parasite GS-specific polyclonal sera was used at 1:250 dilution. FITC-conjugated donkey anti-mouse IgG (Thermo Fisher Scientific, A24501) was used at 1:250 dilution. Images were captured with 100×objective using Olympus IX83 microscope with DP73 high-performance camera.

### Example 5

### Generation of PfGS^{cKS} Parasites and Performing Conditional Knock Sideways

For generating PfGS^{cKS} parasites, C-terminal portion of PfGS representing 1245-2069 bp (without stop codon at the end) was cloned into pSLI-2xFKBP-GFP plasmid for in-frame fusion with FKBP and GFP. The following were the forward (F) and reverse (R) primers used: PfGS^{cKS} (F) of Sequence ID No. 3: 5′-GCATGCGGCCGCTAAATATTCTATCATAATGATCCTTCTACTTTC-3′ and PfGS^{cKS} (R) of Sequence ID No. 4: 5′-CGATCCTAGGACATTCATAATATAAGTGATAATCATAAGC-3′. The restriction sites are underlined. 50 μg of the plasmid construct was nucleofected into purified mature schizonts of Pf3D7 using P3 primary cell 4D-Nucleofector™ X Kit L (Lonza, V4XP-3024) and the parasites having episomal plasmid were selected after 24 h with 4 nM WR99210 (Jacobus Pharmaceutical Company Inc.). When the parasitemia reached around 2%, selection of the integrated parasites was carried out by the addition of 400 μg G418 per ml of culture volume (Gibco, 1181031). The in-frame fusion of GS with FKBP and GFP in the selected parasites was confirmed by genomic DNA PCR, RT-PCR, Western and fluorescence analyses. To mislocalize PfGS by conditional knock sideways, PfGS^{cKS} parasites were nucleofected with 50 μg of mislocalizer pLyn-FRB-mCherry plasmid, followed by selection with 2 μg of Blasticidin-S (Thermo Fisher Scientific, R21001) per ml of culture volume. The presence of mislocalizer plasmid was confirmed by examining the mCherry fluorescence in live parasites and its membrane localization. The mislocalization of PfGS was induced by the addition of 250 nM rapamycin (Sigma-Aldrich, R0395) and verified by the translocation of GFP fluorescence from the parasite cytosol to plasma membrane in live parasites.

### Example 6

### MSO and PPT Inhibition Studies in Pf

In vitro inhibition studies were carried out either in RPMI-1640 lacking L-glutamine (RPMI^{-gln}) or RPMI-1640 medium containing physiological concentrations (0.5 mM) of L-glutamine (RPMI^{Pgln}). MSO/PPT treatment was carried out in synchronized Pf cultures containing late rings and early trophozoites and the parasite growth was monitored for 24 h and 48 h by examining Giemsa-stained smears and by performing flow cytometry and ³H-hypoxanthine uptake. ³H-hypoxanthine was added to the cultures after 3 h of MSO/PPT addition.

### Example 7

### Protein Labelling

In vitro protein labelling studies for MSO/PPT treated Pf cultures were carried out by adding 100 μCi of ³⁵S-Methionine and Cysteine mix (Invivo ProTwin Label (LCS-8), BRIT) to 4 ml culture volume at 3 h post-addition of MSO/PPT. After 9 h of radiolabelling, parasites were isolated by saponin lysis, resuspended in 50 mM Tris pH 8.0 containing 100 mM NaCl, 2% glycerol and 0.1% Triton X-100, and lysed by sonication. The lysates were resolved on 10% SDS-PAGE gel and phosphorimager scanning was carried out to examine the radiolabelling of proteins using Amersham Typhoon 5 Biomolecular Imager. In parallel, 10 μl of the lysates were spotted on Whatman filter paper grade I, dried and washed subsequently with hot and cold 10% (w/v) trichloroacetic acid to remove free amino acids and charged-tRNAs, followed by diethyl ether to remove lipids. The filter papers were then placed in Ultima Gold XR scintillation cocktail and the radioactive counts were measured using MicroBeta² Microplate Counters. All these experiments were performed with identical parasitemia and hematocrit between the untreated and treated groups by equally splitting the infected cultures.

### Example 8

### Estimation of Aspartate, Glutamate, Asparagine and Glutamine Levels in Pf Parasites

The levels of aspartate, glutamate, asparagine and glutamine in Pf pellets treated with MSO were estimated by HPLC as well as by LC-MS/MS. 10 ml of synchronized Pf cultures having ˜2% parasitemia were treated with MSO for 12 h when the parasites were predominantly in late rings and early trophozoites, followed by saponin lysis to isolate the parasites. Parasite pellets were then extracted with 10 volumes of 50% methanol in water (v/v), followed by lyophilisation and solubilizing the lyophilized extracts with 50 μl of water. For HPLC, the extracts were derivatized with OPA, separated on Poroshell 120 HPH-C18 column (4.6 mm×100 mm×2.7 μm) using Agilent 1260 Infinity HPLC System as mentioned for the recombinant protein assays. The peak areas of the MSO-treated samples with respect to the untreated controls was used to measure the fold changes of aspartate, glutamate, asparagine and glutamine. To compensate the variations that arise because of the changes in parasite yield, the data were normalized with the average fold changes of the peak areas obtained for at least five different amino acids—arginine, serine, histidine, threonine and tyrosine that showed unambiguous separation in HPLC. For LC-MS/MS, the parasite extracts were derivatized with 6-aminoquinolyl-N-hydroxysuccinimidyl carbamate (AQC), cleaned up with SPE and dried under vacuum, followed by reconstitution with 50 μl of 0.5% acetonitrile containing 0.1% formic acid. 10 μl of the sample was used for LC-MS/MS analysis carried out in Acquity C18 column (1.8 μm, 2.1 mm×100 mm) using Dionnex Ultimate3000 UHPLC system coupled with Q Exactive mass spectrometer (Thermo Fisher Scientific). Mobile phase consisted of Solvent A: 10 mM ammonium acetate containing 0.1% formic acid and solvent B: acetonitrile containing 0.1% formic acid. Mass spectrometry was performed under spray voltage of 4000/2500V with vaporizer temperature of 250° C., sheath gas and auxillary gas flow rate of 30 and 10 Arb, respectively. The acquisition was performed in Parallel Reaction Monitoring mode at 35000 resolution with a normalized collision energy of 25 eV. The calibration curves were prepared for the individual amino acids and the samples were spiked with internal standards containing deuterated amino acids. The fold changes of aspartate, glutamate, asparagine and glutamine was calculated based on their relative abundances in MSO-treated and untreated controls, after normalizing with the average fold changes of the relative abundancies obtained for arginine, serine, histidine, threonine and tyrosine.

### Example 9

eIF2α Phosphorylation in Pf Parasites

Pf3D7 cultures were subjected to two rounds of tight synchronization within a span of 3 h in the previous cycle, followed by one additional synchronization in the subsequent cycle immediately after RBC invasion. After 3 h post-invasion, ring stage parasites were treated for 6 h with 250 μM MSO in RPMI^{-gln} medium. The parasite pellets were then prepared by saponin treatment and lysed with 0.5× PBS containing 0.5% Triton X-100, Halt protease inhibitor and PhosSTOP (Roche, 4906845001). Western blot analysis was performed with phosphorylated elF2α (#3398, Cell Signaling Technology) and total elF2α (#9722, Cell Signaling Technology) antibodies.

### Example 10

### Inhibition Studies With Pf Clinical Samples

The collection of clinical samples was carried out by withdrawing blood from febrile patients. Pf infections were confirmed by examining Giemsa-stained thick and thin blood smears under light microscope, and by performing rapid diagnostic test and PCR for 18S rRNA. The infected blood was collected in heparinized vacutainers and the subsequent procedures were carried out in BSL-2 facility under sterile conditions. The infected blood was centrifuged to remove the plasma and buffy coat, followed by two washes with RPMI-1640 medium containing 10% heat-inactivated human O⁺ serum. The packed cells were then resuspended in the same medium and the hematocrit was adjusted to 5%. The cultures were treated with different concentrations of MSO/PPT and ³H-hypoxanthine addition was carried out after 3 h of MSO/PPT addition to assess the parasite growth. The cultures were incubated following the standard conditions of Pf maintenance and the experiments were performed for each clinical isolate in RPMI^{-gln} as well as in RPMI^{Pgln}. All these experiments were typically carried out in a final culture volume of 1.2 ml for the respective concentrations of MSO or PPT. Parasite growth assessment was also verified by examining the Giemsa-stained smears.

### Example 11

### Ring-Stage Survival Assays With ART-Resistant PfCam3.I^{R539T} Strain

PfCam3.I^{R539T} strain was maintained under standard conditions. When the parasitemia reached around 2% with predominant ring stages, the cultures were subjected to two rounds of tight synchronization within a span of 3 h in the previous cycle, followed by one additional synchronization in the subsequent cycle when they are in early rings. The parasitemia was adjusted to 0.5-1.0% in 2% hematocrit and ring-stage survival assays were carried out by the addition of 700 nM ART (Sigma-Aldrich, 361593) or DHA (Sigma-Aldrich, D7439). For the combination of ART with MSO/PPT and DHA with MSO/PPT, MSO or PPT was added along with ART/DHA. The exposure of rings to ART/DHA or ART/DHA in combination with MSO/PPT was precisely carried out for 6 h, followed by the removal of supernatant and washing the infected-RBCs twice with RPMI-1640 medium. The infected RBCs were then resuspended in RPMI-1640 medium and maintained continuously. The medium change was carried out at 48 and 72 h, and after 96 h, the culture parasitemia was examined by Giemsa-stained smears and flow cytometry. For growth assessment by ³H-hypoxanthine uptake, the label was added at 72 h and the cells were harvested at 96 h. The experiments were carried out independently in RPMI^{-gln} and RPMI^{Pgln} medium.

### Results

### PfGS Activity and Inhibition

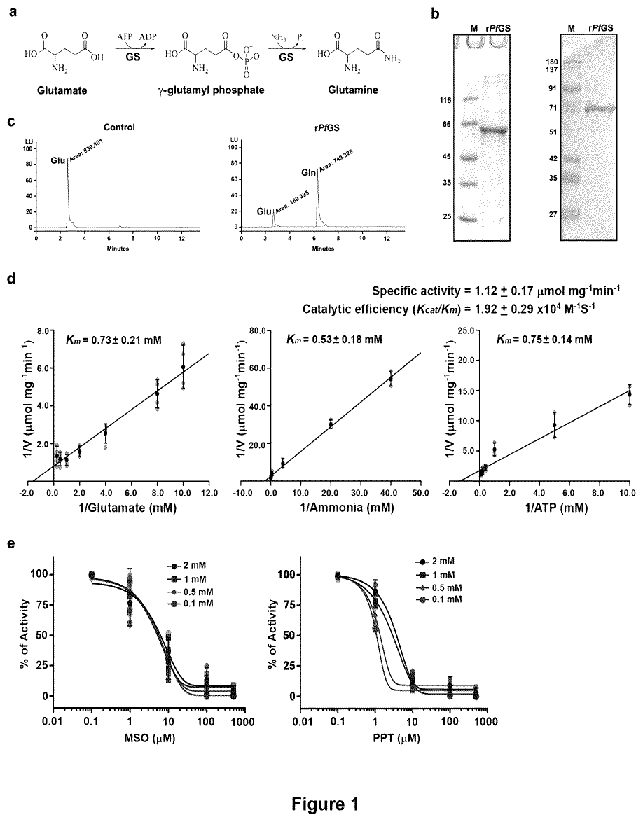

GS catalyzes the synthesis of glutamine by incorporating ammonia into glutamate with concomitant ATP hydrolysis. In the presence of divalent cations such as Mg²⁺ or Mn²⁺, the terminal phosphate from ATP is transferred to glutamate resulting in γ-glutamyl phosphate intermediate, which is subsequently attacked by ammonia to form glutamine. The recombinant (r) PfGS with N-terminal His-tag was overexpressed in E. coli and purified using Ni²⁺-NTA resin. The purified rPfGS showed a molecular weight of around ˜65 kDa in SDS-PAGE and reacted with anti his-tag antibody. HPLC analyses of the purified rPfGS incubated with glutamate and ammonia in the presence of ATP and MgCl₂ showed the formation of glutamine suggesting that rPfGS is enzymatically active. The specific activity and catalytic efficiency (K_{cat}/Kₘ) were found to be 1.12±0.17 μmol mg⁻¹min⁻¹ and 1.92±0.29×10⁴ M⁻¹S⁻¹ for rPfGS. L-methionine sulfoximine (MSO) is a potent irreversible inhibitor of GS. MSO-phosphate generated through the phosphorylation of MSO by GS serves as a transition state analogue that binds non-covalently and stabilizes the flexible loop of GS active site thereby, preventing glutamate entry. In addition, the methyl group of MSO-P occupies the ammonium binding site to prevent further reaction. Inhibition studies carried out with recombinant enzyme showed that rPfGS is sensitive for MSO inhibition with Kᵢ value of 5.64±0.39 μM. Phosphinothricin (PPT; also known as glufosinate), widely used as a broad-spectrum herbicide, is another potent irreversible inhibitor of GS whose mechanism of inhibition is similar to that of MSO. rPfGS is also sensitive to PPT with a Kᵢ value of 2.31±1.10 μM. These data indicated that the parasite GS can be inhibited by the transition state analogues (FIG. 1).

### Inhibition of Pf Growth by MSO and PPT

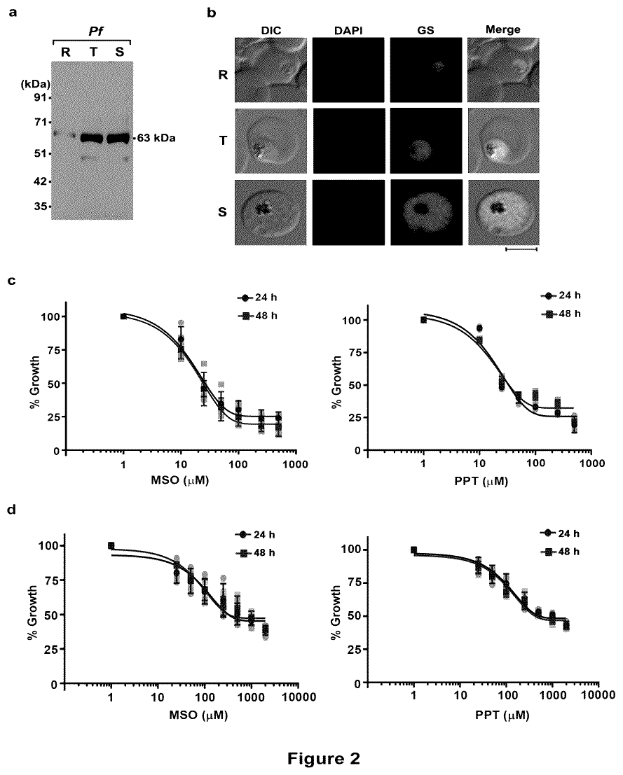

Western analyses carried out for Pf lysates using PfGS antibodies showed GS expression in all the asexual stages. Immunofluorescence analyses showed an abundant cytosolic localization wherein, the fluorescence signal was observed all over the parasite in rings, trophozoites and schizonts. The results obtained with rPfGS were further validated by examining the effect of MSO and PPT on in vitro growth of Pf. It is known that the presence of glutamine in the culture medium can compete with the cellular uptake of MSO and PPT because of its structural similarity. Therefore, the effect of MSO and PPT on in vitro growth of Pf was examined in the presence and absence of glutamine. Both, MSO and PPT addition could inhibit the growth of Pf asexual stages in the presence and absence of glutamine. The IC₅₀ values for MSO and PPT inhibiting Pf growth in RPMI medium lacking glutamine (RPMI^{-gln}) were in the range of ˜30 μM and the Giemsa-stained smears showed the presence of stressed, arrested, pyknotic and dead parasites. In RPMI medium containing physiological levels of (0.5 mM) glutamine (RPMI^{Pgln}), the IC₅₀ values obtained for MSO and PPT were almost 10 times higher. The results from the chemical inhibition studies suggested the potential of targeting GS for Pf infections (FIG. 2).

GS is Required for the Development of Asexual Stages of P. falciparum

The requirement of GS for the growth of Pf parasite is established using a genetic approach. We generated a conditional, mislocalizing, knock sideways (cKS) 3D7 strain for GS (PfGS^{cKS}) through selection-linked integration approach wherein, GS was fused in-frame with FKBP (FK506 binding protein) and GFP through linkers, followed by a neomycin selectable marker separated by skip peptide. PfGS^{cKS} parasites displayed 60% reduction of growth in RPMI^{Pgln} when compared with Pf3D7 parasites, on day 7 representing three asexual cycles. Similar results were also obtained with normal RPMI-1640 medium containing 2 mM glutamine (RPMI^{Ngln}). Moreover, Pf3D7 cultures could be maintained continuously for several days in RPMI^{-gln}. Unlike Pf3D7, PfGS^{cKS} parasites failed to grow in RPMI^{-gln} and no viable parasites could be detected after 4 days of glutamine removal. All these results indicated that intervention of endogenous GS activity can drastically affect the growth of Pf asexual stages, highlighting the significance of GS in Pf.

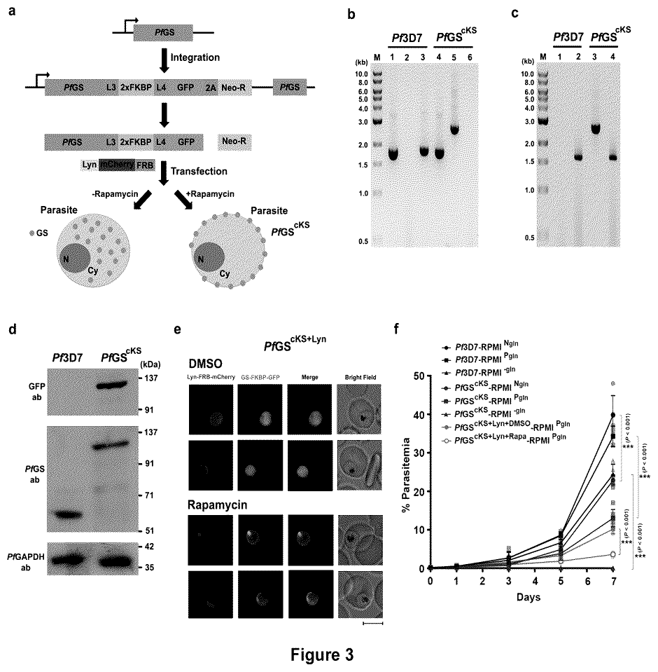

To perform cKS, PfGS^{cKS} strain was transfected with a plasmid that led to the expression of plasma membrane targeting Lyn peptide fused with FRB and mcherry. After selecting the transfected PfGS^{cKS} parasites (PfGS^{cKS+Lyn}) with blasticidin, GS was knocked sideways from cytosol to the plasma membrane by the addition of rapamycin and this was evident from the change in the localization of GFP signal. The rapamycin-induced mislocalization of GS from its site of action caused further reduction in PfGS^{cKS} parasite growth, leading to almost 90% decrease in the parasite growth in RPMI^{Pgln} when compared with Pf3D7 parasites, clearly indicating the significance of GS in the asexual stages of Pf. These results also suggested that the Hb-derived and extracellular glutamine are inadequate for the optimal growth of Pf (FIG. 3).

### Targeting GS Affects Pf Protein Synthesis

To gain insights on the requirement of GS in Pf, the metabolic labelling of proteins using [³⁵S]-Methionine and-Cysteine in in vitro cultures of Pf was examined. MSO and

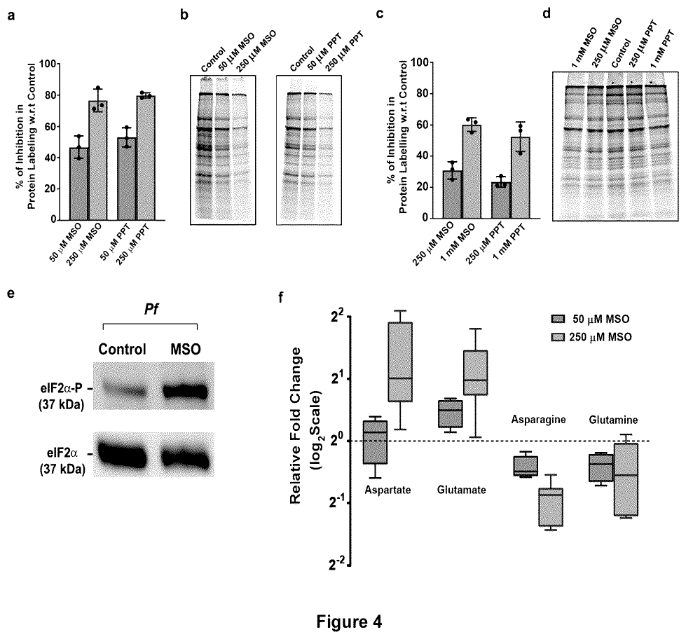

PPT treatments performed for 12 h could inhibit protein synthesis in Pf. While a substantial 40-50% inhibition in protein synthesis of Pf was observed at 50 μM MSO and PPT, the inhibition was close to 80% at 250 μM MSO and PPT in RPMI^{-gln} medium. In RPMI^{Pgln} medium, MSO and PPT could lead to ˜50% inhibition in protein synthesis at 1 mM concentration. A careful examination of free glutamine levels in Pf parasites treated with MSO in RPMI^{-gln} medium with respect to the untreated parasites did not show a significant decrease in the glutamine levels. However, the free asparagine levels were reduced to the extent of 1.5-2.0 fold in Pf parasites treated with MSO with respect to the untreated control. This in turn suggested that the inhibition in protein synthesis observed for Pf parasites is mainly due to the requirement of glutamine for asparagine synthesis. It is also known that Pf proteins are rich in asparagine. Further, it was examined whether GS inhibition by MSO in Pf can lead to eIF2α phosphorylation—a molecular signature of amino acid deprivation leading to the inhibition of protein synthesis. Interestingly, a short-term exposure of in vitro cultures to 250 μM MSO for 6 h could lead to a prominent phosphorylation of eIF2α in Pf. These results suggested that inhibition of parasite protein synthesis and the requirement of asparagine arising due to the asparagine-rich nature of Pf proteins are responsible for the requirement of GS in Pf (FIG. 4).

### GS Inhibitors Affect the Growth of Pf Clinical Isolates and ART-Resistant Parasites

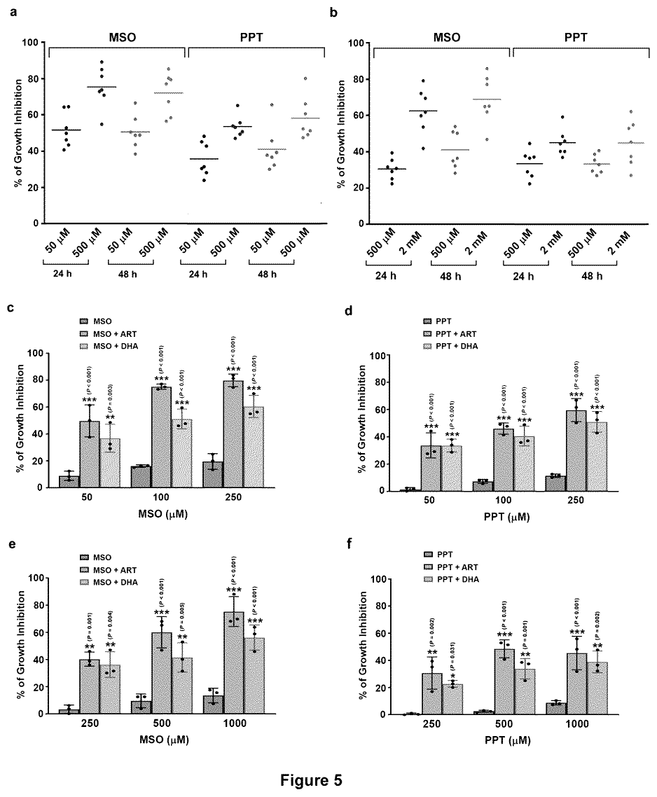

Blood samples collected from Pf-infected patients were incubated in in vitro cultures without or with glutamine and the parasite growth was assessed in the presence of MSO and PPT. MSO and PPT could inhibit the growth of Pf clinical samples to the extent that was observed for Pf3D7 strain. To examine whether GS can be targeted for artemisinin (ART) resistance, ring-stage survival assay (RSA) using tightly synchronized rings of ART-resistant PfCam3.I^{R539T} strain was performed. ART or dihydroartemisinin (DHA; 700 nM) exposure for 6 h resulted in viable parasites for PfCam3.I^{R539T} cultures after 72 h, but not in ART-sensitive Pf3D7 cultures. Interestingly, the exposure of ART or DHA in combination with MSO for 6 h led to ˜50-80% reduction in viable parasites at 50-250 μM concentrations of MSO in RPMI^{-gln} in vitro cultures with respect to the treatment with ART or DHA alone. The exposure of PfCam3.I^{R539T} rings to MSO alone for 6 h could only lead to 10-20% inhibition with respect to the untreated control. A similar inhibition pattern was observed in RPMI^{+Pgln} in vitro cultures wherein, 50% inhibition was observed at ˜500 μM MSO. PPT could also reduce the viability of PfCam3.I^{R539T} rings in combination with ART or DHA, although PPT was slightly less effective than MSO. These data suggested the potential of targeting GS to tackle ART-resistance in Pf by using a combination of MSO/PPT with ART/DHA (FIG. 5). It is to be noted that ring-survival assays were carried out for the viability of artemisinin-resistant parasites that emerge after artemisinin (ART) or dihydroartemisinin (DHA) exposure. Therefore, the percentage of inhibition for ART/DHA with MSO/PPT (GS inhibitors) has been represented with respect to ART/DHA alone. As evident from FIG. 5, the inclusion of MSO/PPT with ART/DHA leads to the inhibition of ART-resistant parasite emergence.

### REFERENCES

1) van der Pluijm, R. W., Amaratunga, C., Dhorda, M. & Dondorp, A. M., Triple artemisinin-based combination therapies for malaria—A new paradigm? Trends Parasitol. 37, 15-24, (2021).
2) Gardner, M. J. et al. Genome sequence of the human malaria parasite Plasmodium falciparum. Nature. 419, 498-511 (2002).
3) Payne, S. H. & Loomis, W. F. Retention and loss of amino acid biosynthetic pathways based on analysis of whole-genome sequences. Eukaryot. Cell. 5, 272-276 (2006).
4) Olszewski, K. L. & Llinás, M. Central carbon metabolism of Plasmodium parasites. Mol. Biochem. Parasitol. 175, 95-103 (2011).
5) Krishnan, A. & Soldati-Favre, D. Amino Acid Metabolism in Apicomplexan Parasites. Metabolites. 11, 61 (2021).
6) Francis, S. E., Sullivan, D. J. Jr. & Goldberg, D. E. Hemoglobin metabolism in the malaria parasite Plasmodium falciparum. Annu. Rev. Microbiol. 51, 97-123 (1997).
7) Babbitt, S. E. et al. Plasmodium falciparum responds to amino acid starvation by entering into a hibernatory state. Proc. Natl. Acad. Sci. U.S.A. 109, E3278-3287 (2012).
8) Cruzat, V., Macedo Rogero, M., Noel Keane, K., Curi, R. & Newsholme, P. Glutamine: Metabolism and Immune Function, Supplementation and Clinical Translation. Nutrients. 10(11), 1564 (2018).
9) MacRae, J. I. et al. Mitochondrial metabolism of sexual and asexual blood stages of the malaria parasite Plasmodium falciparum. BMC Biol. 11, 67 (2013).
10) Mack, S. R., Samuels, S. & Vanderberg, J. P. Hemolymph of Anopheles stephensi from uninfected and Plasmodium berghei-infected mosquitoes. 2. Free amino acids. J. Parasitol. 65, 130-136 (1979).
11) PlasmoDB. Plasmodium Genomics Resource. https://plasmodb.org/plasmo/app
12) Bunditvorapoom, D. et al. Fitness loss under amino acid starvation in artemisinin-resistant Plasmodium falciparum isolates from Cambodia. Sci. Rep. 8, 12622 (2018).
13) Mok, S. et al. Artemisinin-resistant K13 mutations rewire Plasmodium falciparum's intra-erythrocytic metabolic program to enhance survival. Nat. Commun. 12, 530 (2021).

1. A pharmaceutical composition for combating drug resistance in Plasmodium falciparum, the composition comprising:
(i) An inhibitor of glutamine synthetase enzyme
(ii) Artemisinin or dihydroartemisinin.

2. The composition as claimed in claim 1, wherein the inhibitor of glutamine synthetase enzyme is in the range of 50 μM to 2 mM in in vitro Plasmodium falciparum cultures and shall be present in the range of 0.1-100 mg/kg of the total body weight in the pharmaceutical composition.

3. The composition as claimed in claim 1, wherein the inhibitor of glutamine synthetase enzyme is selected from L-Methionine sulfoximine or Phosphinothricin.

4. The composition as claimed in claim 1, wherein the inhibitor of glutamine synthetase irreversibly inhibits glutamine synthetase activity leading to a decrease in the production of asparagine and protein synthesis.

5. A method of suppressing of the growth of Plasmodium falciparum, the method comprising targeting glutamine synthetase and asparagine requirement by providing a pharmaceutical composition as claimed in claim 1 comprising an inhibitor of glutamine synthetase enzyme and artemisinin or dihydroartemisinin.

6. The method as claimed in claim 5, wherein the inhibitor of glutamine synthetase enzyme is in the range of 50 μM to 2 mM in in vitro Plasmodium falciparum cultures and shall be present in the range of 0.1-100 mg/kg of the total body weight in the pharmaceutical composition.

7. The method as claimed in claim 5, wherein the inhibitor of glutamine synthetase enzyme is selected from L-Methionine sulfoximine or Phosphinothricin.

8. The method as claimed in claim 5, wherein the inhibitor of glutamine synthetase irreversibly inhibits glutamine synthetase activity leading to a decrease in the production of asparagine and protein synthesis.

9. A method of treating malaria and combating artemisinin resistance, by targeting glutamine synthetase and asparagine requirement using glutamine synthetase inhibitors as claimed in claim 1 along with the existing artemisinin-based combination therapies selected from artesunate and amodiaquine; artesunate and mefloquine; artesunate and sulfadoxine-pyrimethamine; artemether and lumefantrine; dihydroartemisinin and piperaquine; artesunate and pyronaridine and/or other antimalarials.

10. Glutamine Synthetase inhibitors for use in the method of suppressing the growth of Plasmodium falciparum as claimed in claim 5.

11. The glutamine synthetase inhibitors as claimed in claim 10, wherein the inhibitors are selected from L-Methionine sulfoximine or Phosphinothricin.

12. Glutamine Synthetase inhibitors for use in the method of suppressing the growth of Plasmodium falciparum as claimed in claim 6.

13. Glutamine Synthetase inhibitors for use in the method of suppressing the growth of Plasmodium falciparum as claimed in claim 7.

14. Glutamine Synthetase inhibitors for use in the method of suppressing the growth of Plasmodium falciparum as claimed in claim 8.

15. Glutamine Synthetase inhibitors for use in the method of preventing and treating malaria and combating artemisinin resistance as claimed in claim 9.


SEQUENCE LISTING (4 sequences)

SEQ ID NO: 1 - RNA, 39 nt, synthetic construct
  source 1..39: mol_type = other RNA
gccaggatccatgaagtccgtgagtttttcaaataatgc

SEQ ID NO: 2 - RNA, 44 nt, synthetic construct
  source 1..44: mol_type = other RNA
gcccagatctctaacattcataatataagtgataatcataagcg

SEQ ID NO: 3 - RNA, 45 nt, synthetic construct
  source 1..45: mol_type = other RNA
gcatgcggccgctaaatattctatcataatgatccttctactttc

SEQ ID NO: 4 - RNA, 40 nt, synthetic construct
  source 1..40: mol_type = other RNA
cgatcctaggacattcataatataagtgataatcataagc